(19) United States
(12) Patent Application Publication
(10) Pub. No.: US 2007/0123751 A1
(43) Pub. Date: May 31, 2007

(54) ENDOSCOPE WITH BRIGHTNESS ADJUSTMENT FUNCTION

(75) Inventor: Tadashi Takahashi, Saitama (JP)

Correspondence Address: GREENBLUM & BERNSTEIN, P.L.C., 1950 ROLAND CLARKE PLACE, RESTON, VA, 20191, US

(73) Assignee: PENTAX CORPORATION, Tokyo (JP)

(21) Appl. No.: 11/563,853

(22) Filed: Nov. 28, 2006

(30) Foreign Application Priority Data
Nov. 29, 2005 (JP) P2005-343461
Nov. 29, 2005 (JP) P2005-343531
Nov. 29, 2005 (JP) P2005-343660
Nov. 29, 2005 (JP) P2005-343704

(51) Int. Cl. A61B 1/06

(52) U.S. Cl. 600/180; 600/118

(57) ABSTRACT

An electronic endoscope has a video-scope with an image sensor, a light source that radiates illuminating-light on an object, and a brightness adjuster that maintains a brightness of an object image at a proper brightness on the basis of an amount of illuminating-light. The electronic endoscope has a brightness state detector and a reporting processor. The brightness state detector detects whether the amount of illuminating-light is aberrant while the brightness is adjusted by the brightness adjuster. The reporting processor reports an aberrant state of the illuminating light.

### BACKGROUND OF THE INVENTION

1. Field of the Invention

The present invention relates to an endoscope that observes an observed portion, such as body cavity. In particular, it relates to illuminating-light irradiated from a light source such, as a lamp.

2. Description of the Related Art

In an endoscope with a brightness adjustment function, a light-amount adjustment mechanism or an electronic shutter function adjusts a brightness of an object image that is displayed on a display. In an electronic endoscope with a video-scope, a luminance of the object image is detected on the basis of image-pixel signals, which are read from a CCD provided in the video-scope. Then, an opening-degree of a stop or an electronic shutter speed is adjusted such that the displayed object image is maintained at a proper brightness. Also, in an electronic endoscope with a self-monitoring or self-diaqnosis function, a system for monitoring a status of the endoscope operation is installed in the electronic endoscope. When electric power is supplied to the endoscope by operating a power button, the system detects whether a trouble, resulting in erroneous operation, occurs in an electronic circuit. If any trouble or aberrant state is detected, warning information is displayed on the monitor.

### SUMMARY OF THE INVENTION

An object of the present invention is to provide an endoscope that is capable of detecting and reporting unusual or aberrant state of the endoscope while the endoscope is used.

An electronic endoscope according to the present invention has a video-scope with an image sensor, a light source that radiates illuminating-light on an object, and a brightness adjuster that maintains a brightness of an object image at a proper brightness by adjusting an amount of illuminating-light. Further, the electronic endoscope has a brightness state detector and a reporting processor. The brightness state detector detects whether the amount of illuminating-light is aberrant, while the brightness is adjusted by the brightness adjuster. The reporting processor informs or reports an aberrant state of the illuminating light when the aberrant state id detected.

According to another aspect of the present invention, an apparatus for diagnosing an electronic endoscope has a brightness state detector that detects whether an amount of illuminating-light is aberrant, while an brightness adjuster maintains a brightness of an object image at a proper brightness on the basis of an amount of illuminating-light; and a reporting processor that reports an aberrant state of the illuminating light.

According to another aspect of the present invention, a method for diagnosing an electronic endoscope includes i) detecting whether an amount of illuminating-light is aberrant while maintaining a brightness of an object image at a proper brightness by adjusting the illuminating-light; and ii) reporting an aberrant state of the illuminating light.

### BRIEF DESCRIPTION OF THE DRAWINGS

The present invention will be better understood from the description of the preferred embodiments of the invention set forth below together with the accompanying drawings, in which:

FIG. 1 is a block diagram of an electronic endoscope according to a first embodiment;

FIG. 2 is a flowchart of a main process performed by the system control circuit;

FIG. 3 is a view showing a subroutine of Step S102 in FIG. 2;

FIG. 4 is a view showing a subroutine of step s103 in FIG. 2;

FIG. 5 is a flowchart of an auto-brightness adjustment process and a self-diagnosis process;

FIG. 6 is a flowchart of a usage state detection process;

FIG. 7 is a block diagram of an electronic endoscope according to a second embodiment;

FIG. 8 is a flowchart of a main routine according to the second embodiment;

FIG. 9 is a flowchart of an auto-brightness adjustment process and a self-diagnosis process according to the second embodiment;

FIG. 10 is a block diagram of an electronic endoscope according to a third embodiment;

FIG. 11 is a view showing a main routine according to the third embodiment;

FIG. 12 is a view showing a flowchart of an auto-brightness adjustment process and a self-diagnosis process according to the third embodiment;

FIG. 13 is a flowchart of a usage state detection process according to the third embodiment;

FIG. 14 is a flowchart of an auto brightness adjustment process and a self-diagnosis process according to a fourth embodiment;

FIG. 15 is a view showing a main routine according to the fourth embodiment;

FIG. 16 is a view showing a subroutine of Step S1302 shown in FIG. 15;

FIG. 17 is a view showing a subroutine of Step S1303 shown in FIG. 15;

FIG. 18 is a block diagram of an electronic endoscope according to a fifth embodiment;

FIG. 19 is a view showing a main routine according to the fifth embodiment; and

FIG. 20 is a view showing a subroutine of Step S1504 shown in FIG. 19.

### DESCRIPTION OF THE PREFERRED EMBODIMENTS

Hereinafter, the preferred embodiments of the present invention are described with reference to the attached drawings.

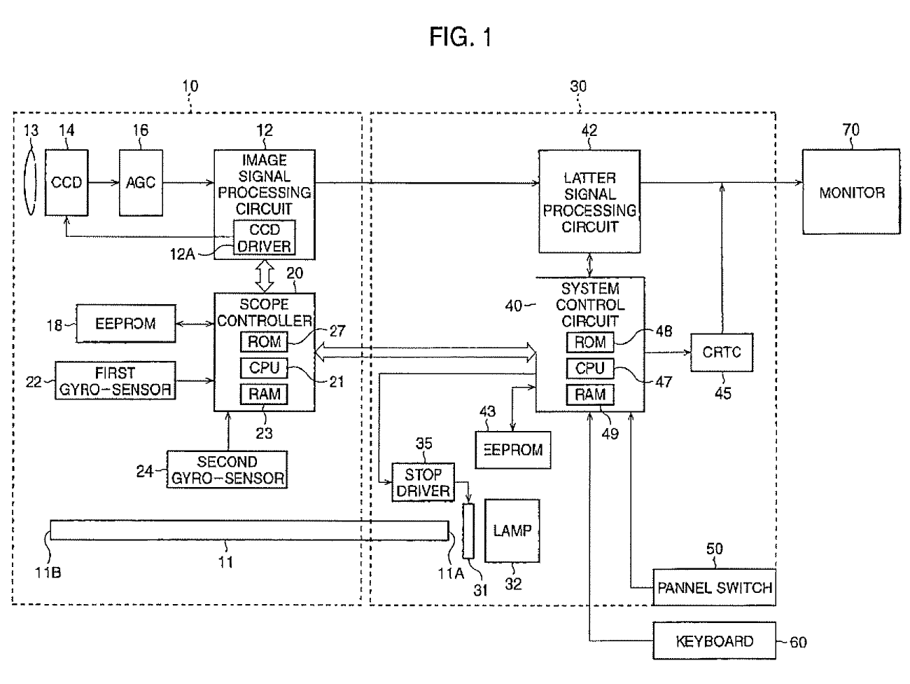

FIG. 1 is a block diagram of an electronic endoscope according to a first embodiment,

The electronic endoscope has a video-scope 10 with a CCD 14, and a video-processor 30. The video-scope 10 is removably connected to the video-processor 30; and a keyboard 60 and a monitor 70 are connected to the video-processor 30. When the video-scope 10 is connected to the video-processor 30, electric power is supplied from the video-processor 30 to the video-scope 10, which allows the video-scope 10 to operate.

When a lamp switch button (not shown) is turned ON, a lamp 32 radiates illuminating light. The illuminating light emitted from the lamp 32 enters the incident surface 11A of a light-guide 11 via a stop 31 and a collecting lens (not shown). The light-guide 11 is constructed of a fiber-optic bundle for directing the illuminating light to a tip end of the video-scope 10. The light exits from the distal end portion 11B of the light-guide 11, and illuminates an observed object via a diffusion lens (not shown).

Light, reflected off the object, reaches the CCD 14 via an objective lens 13, so that an object image is formed on the photo-sensitive area of the CCD 14. A color filter (not shown), checkered by four color elements of Yellow (Y), Magenta (M), Cyan (C), and Green (G), is arranged on the photo-receiving area such that the four color elements are opposite to pixels arranged in the photo-sensitive area. Based on the light passing through each color element, analog image-pixel signals are generated by the photoelectric transformation effect.

The generated image-pixel signals are read from the CCD 14 to an image signal processing circuit 12 via an AGC (Auto Gain Control) circuit 16, at regular time intervals in accordance with clock pulse signals output from a CCD driver 12A in the video signal processing circuit 12. The NTSC (or PAL) standard is herein applied as the TV standard; therefore, the image-pixel signals corresponding to one field image are read from the CCD 14 at a 1/60 (or 1/50) second intervals.

In the image signal processing circuit 12, various processes, such as a gamma correction process, a white balance process, and so on, are carried out on the image-pixel signals, so that luminance and color difference signals are generated. The generated luminance and color difference signals are output to the video-processor 30. In a latter signal processing circuit 42, video signals are generated from the luminance and color difference signals, and are output to the monitor 70 so that an observed image is displayed on the monitor 70.

The video-scope 10 has a scope controller 20 including a CPU 21, a RAM 23, and a ROM 27; and controls an operation of the video-scope 10 by outputting control signals to circuits in the video-scope 10. In the ROM 27, a program associated with a process of the video-scope 10 is stored. On the other hand, in an EEPROM 18, data associated with characteristics of the video-scope 10 is stored.

The video-processor 30 has a system control circuit 40, which includes a CPU 47, a ROM 48, and a RAM 49; and controls the operation of the video-processor 30. A program for controlling the video-processor 30 is stored in the ROM 48, whereas a data associated with the lamp 32 is stored in an EEPROM 43. The system control circuit 40 outputs character codes to a CRTC (CRT Controller) 45, and the CRTC 45 outputs character signals in accordance with the character codes so that character information is displayed on the monitor 70. The system control circuit 40 and the scope-controller 20 transmit data to each other.

The stop 31 opens and closes to adjust an amount of the illuminating light, and is driven by a stop driver 35. To carry out an auto brightness adjustment process, the system control circuit 40 controls the stop 31 on the basis of luminance signals, which are fed from the latter signal processing circuit 42, so as to maintain the brightness of the displayed observed image at a proper brightness. Concretely, based on a difference between the detected luminance level and a reference luminance level (or the ratio of the detected luminance level to the reference luminance level), the system control circuit 40 adjusts an opening-degree of the stop 31 by outputting control signals to the stop driver 35.

A first gyro-sensor 22 and a second gyro-sensor 24, provided in the video-scope 10, detect whether the video-scope 10 is used by an operator; namely, whether the operator user or manipulates the video-scope 10 for endoscope work, such as for an operation, or for treatment. The first and second gyro-sensors 22 and 24 detect, respectively, two angular velocities with respect to two directions perpendicular to each other, when the video-scope 10 is used.

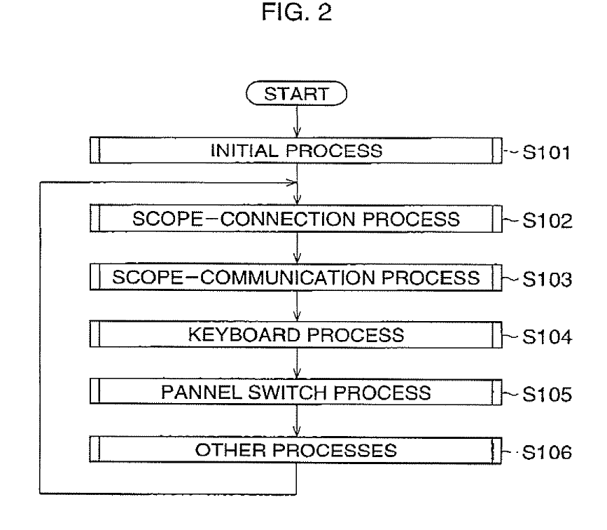

FIG. 2 is a flowchart of a main process performed by the system control circuit 40.

In Step S101, the initial setting process is performed so that variables are set to initial values. In Step S102, a process associated with a connection of the video-scope 10 is performed, and in step S103, a process associated with data communication to the video-scope 10 is performed. In Step S104, a process associated with an operation of the keyboard 60 is performed, and in Step S105, a process associated with an operation on a panel switch 50 is performed. Then, in Step S106, other processes are performed.

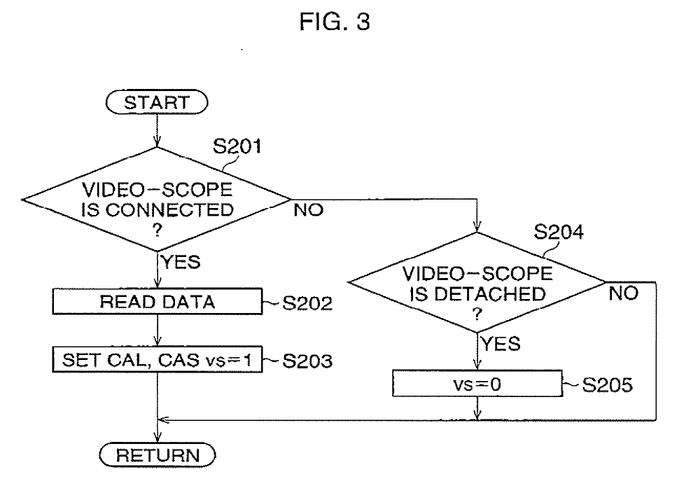

FIG. 3 is a view showing a subroutine or Step S102 in FIG. 2.

In Step S201, it is determined whether the video-scope 10 is newly connected to the video-processor 30. Herein, various video-scopes, corresponding to a lower digestive organ such as the colon, an upper digestive organ such as the stomach, the lungs, or so on, are prepared; and one video-scope is selectively connected to the video-processor 30.

When it is determined in Step S201 that the video-scope 10 is newly connected to the video-processor 30, the process goes to Step S202. In Step S202, data associated with the video-scope 10, such as a registration number, is read from the EEPROM 18. Then, in Step S203, a scope-connection variable “vs” is set to 1. The scope-connection variable “vs” indicates a connection status of the video-scope. The scope-connection variable “vs” is set to 1 when the video-scope 10 is connected to the video-processor 30, whereas the scope-connection variable “vs” is set to 0 when the video-scope 10 is not connected to the video-processor 30. Also, in Step S203, reference ratio or proportions “CAL” and “CAS” associated with large and small opening-degrees, respectively, are set in accordance with the connected video-scope 10, as described later.

On the other hand, when it is determined in Step S201 that the video-scope 10 is not newly connected to the video-processor 30, the process goes to Step S204. In Step S204, it is determined whether the video-scope 10 is detached from the video-processor 30. When it is determined that the video-scope 10 is not detached from the video-processor 30, the process is terminated. On the other hand, when it is determined that the video-scope 10 is detached from the video-processor 30, the process goes to Step S205. In Step S205, the scope-connection variable “vs” is set to 0,

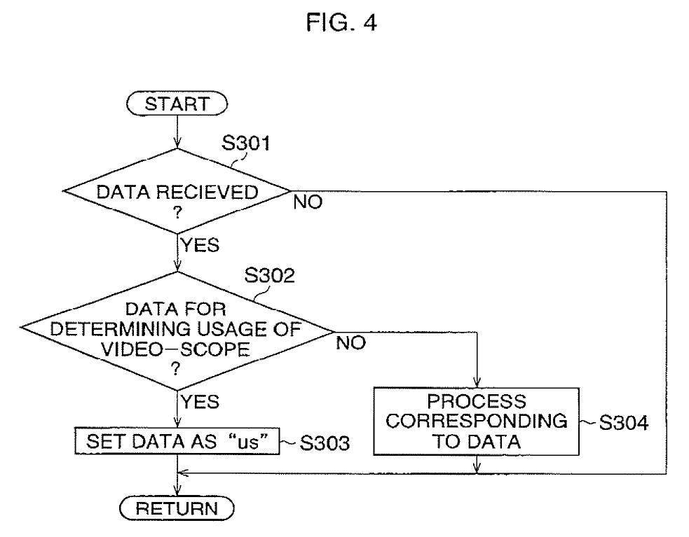

FIG. 4 is a view showing a subroutine of Step S103 in FIG. 2.

In Step S301, it is determined whether data has been transmitted from the video-scope 10. When it is determined that the data has not been transmitted from the video-scope 10, the process is terminated. On the other hand, when it is determined that the data has been transmitted from the video-scope 10, the process goes to step S302. At step S302, it is determined whether the transmitted data is data for determining whether the video-scope 10 is used.

When it is determined that the transmitted data is not data for determining whether the video-scope 10 is used, the process goes to Step S304. At Step S304, a process corresponding to the data is performed. On the other hand, when it is determined that the transmitted data is data for determining whether the video-scope 10 is used, the process goes to Step S303. In Step S303, the value of a usage state variable “us”, described later, is set to the value of the transmitted data.

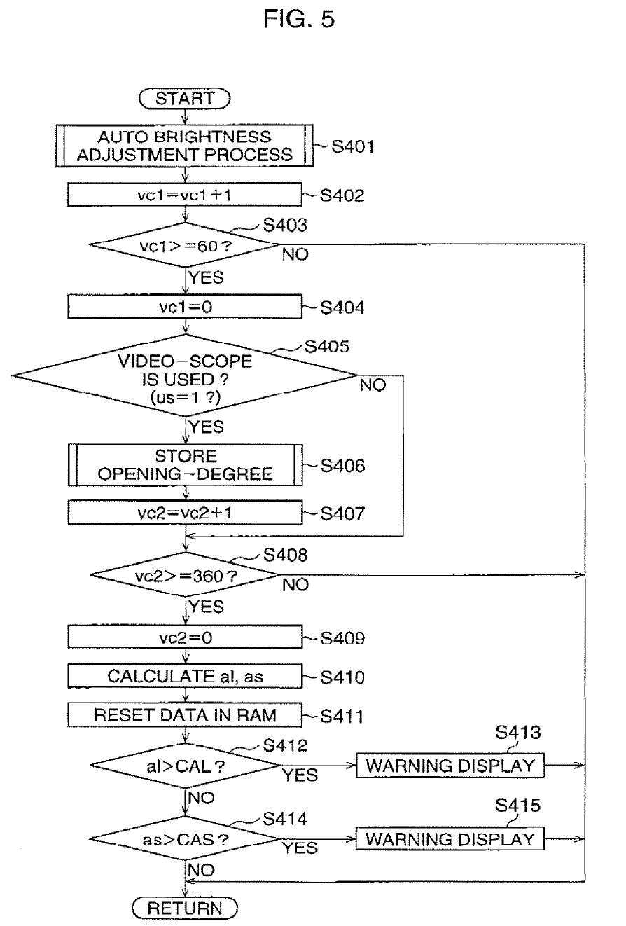

FIG. 5 is a flowchart of an auto-brightness adjustment process and a self-diagnosis process performed by the system control circuit 40. This process is carried out by interrupting in the main routine shown in FIG. 2, at 1/60 second intervals.

In Step S401, the auto brightness adjustment process is performed. Namely based on a luminance difference between a detected luminance level and a reference luminance level indicating a proper brightness of the object, the stop 31 is opened and closed such that the luminance difference does not occur. The stop 31 closes if the detected luminance level is higher than the reference luminance level, while the stop 31 opens if the detected luminance level is smaller than the reference luminance level.

In Step S402, a first recording timer variable “vc1” is incremented by 1. The first recording timer variable “vc1” is a variable that measures an interval for storing an opening-degree of the stop 31 in the RAM 49 at one-second intervals periodically. The first recording timer variable “vcl” is initially set to 0 at Step S101 shown in FIG. 2. In Step S403, it is determined whether the first recording timer variable “vcl” is equal to or more than 60; namely, whether one second has passed.

When it is determined that the first recording timer variable “vc1” is shorter than 60, in Step S403, the process is terminated. On the other hand, when it is determined that the first recording timer variable “vc1” is equal to or greater than 60, the process goes to Step S404. In Step 3404, the first recording timer variable “vc1” is set to 0.

In Step S405, it is determined whether the usage state variable “us” is 1. The usage state variable “us” indicates the used or unused status of the video-scope 10. Herein, it is determined whether the video-scope 10 is substantially used; namely, whether the video-scope 10 is operated for endoscope work, a treatment, an operation, etc. The usage state variable “us” is set to 1 when the video-scope 10 is substantially used, whereas the usage state variable “us” is set to 0 when the video-scope 10 is not substantially used.

When it is determined that the video-scope 10 is not substantially used, the process skips to Step S408. On the other hand, when it is determined that the video-scope 10 is substantially used, the process goes to Step S406. In Step S406, the opening-degree of the stop 31, which is presently set by the system control circuit 40, is temporarily stored in the RAM 49 as data. Concretely speaking, the frequency distribution data of the opening-degree is stored in the RAM 49.

The opening-degree is herein represented by an integer in the range from 0 to 240. The amount of illuminating light increases as the value of the opening-degree increases. The opening-degree is represented by 0 when the stop 31 is fully closed, and the opening-degree is represented by 240 when the stop 31 is fully opened. The range from 0 to 240 of the opening-degree is divided into seven grades or stages; a section from 0 to 39, a section from 40 to 79, a section from 80 to 119, a section from 120 to 159, a section from 160 to 199, a section from 200 to 219, and a section from 220 to 240. The detected opening-degree is assigned to a corresponding grade such that the value of the corresponding frequency is incremented. By successively incrementing the value of the corresponding frequency, the frequency distribution data is generated. After Step S406 is carried out, the process goes to Step S407,

In step S407, a second recording timer variable “vc2” is incremented by 1. The second recording timer variable “vc2” is a variable that measures an interval for carrying out an aberrant status detection process, described later, at six-minute intervals. The second recording timer variable “vc2” is initially set to 0 at Step S102 shown in FIG. 2.

In Step S408, it is determined whether the second recording timer variable “vc2” is equal to or greater than 360; namely, whether six minutes has passed from the previous aberrant status detection process. When it is determined that the second recording timer variable “vc2” is smaller than 360, the process is terminated. on the other hand, when it is determined that the second recording timer variable “vc2” is equal to or greater than 360, the process goes to Step S409, where the second recording timer variable “vc2” is set to 0.

In Step S410, based on the frequency distribution data of the opening-degree, which has been stored in the RAM 49, a first ratio “al” and a second ratio “as” are calculated. Note that, the frequency distribution data is obtained during the six minutes over which the video-scope 10 is substantially used. The first ratio “al” represents a ratio of the number of incidences of relatively large opening-degree to the total detected number of incidences of opening-degrees; namely, the ratio of the detected number of times of the relatively large opening-degree to the total detected number of times during the six minutes (=360). Herein, the first ratio “al” indicates the proportion of the seventh grade; the section from 220 to 240. On the other hand, the second ratio “as” represents a ratio of the number of incidences of relatively small opening-degree to the total detected number of incidences of opening-degrees. Herein, the second ratio “as” indicates the proportion of the first grade; namely, the section from 0 to 39. In Step S411, the frequency distribution data stored in the RAM 49 is reset to 0.

In Step S412, it is determined whether the first ratio “al” exceeds the reference ratio or the large opening-degree “CAL” determined at Step S203 in FIG. 3. Namely, it is determined whether the situation in which the opening-degree of the stop 31 is extremely large continues while the auto-brightness adjustment process is perforated. For example, the amount of illuminating light or the brightness decreases due to dirt on the tip surface of the video-scope 10, or a decrease in light irradiated from the lamp 32. The decrease of illuminating-light results in a state in which a relatively large opening-degree continues. The value of the reference ratio “CAL” is determined in accordance with the type of the video-scope 10. For example, in the case of the video-scope for the bronchi, the reference ratio “CAL” is set to 90 percent; in the case of the video-scope for the stomach, the reference ratio “CAL” is set to 95 percent; in the case of the video-scope for the colon, the reference ratio “CAL” is set to 85 percent.

When it is determined at Step S412 that the first ratio “al” exceeds the reference ratio “CAL”, the process goes to Step S413. In Step S413, character signals are output from the CRTC 45 so as to display character information that reports an aberrant situation of the electronic endoscope to the operator. Thus, the operator can recognize whether the operation or motion of the video-scope 10 or the video-processor is unusual or aberrant with respect to the amount of illuminating-light. On the other hand, when it is determined that the first ratio “al” does not exceed the reference ratio “CAL”, the process goes to Step S414.

In Step S414, it is determined whether the second ratio of the small opening degree “as” exceeds the reference ratio of the small opening-degree “CAS” determined at Step 203 in FIG. 2. For example, when the amount of light radiated from the lamp 32 becomes unexpectedly large due to an accident of the lamp 32, a situation where the value of the opening-degree is extremely small continues during the auto brightness adjustment process, since the stop 31 closes to restrict the extremely large amount of illuminating-light. Herein, in the case of the video-scope for the bronchi, the reference ratio “CAS” is set to 25 percent; in the case of the video-scope for the stomach, the reference ratio “CAS” is set to 25 percent; in the case of the video-scope for the colon, the reference ratio “CAS” is set to 35 percent.

When it is determined that the second ratio of the small opening-degree “as” does not exceed the reference ratio “CAS”, the process is terminated. On the other hand, when it is determined that the second ratio “as” exceeds the reference ratio “CAS”, the process goes to Step S415, where the character signals are output from the CRTC 45 so as to display character information that reports an aberrant situation,

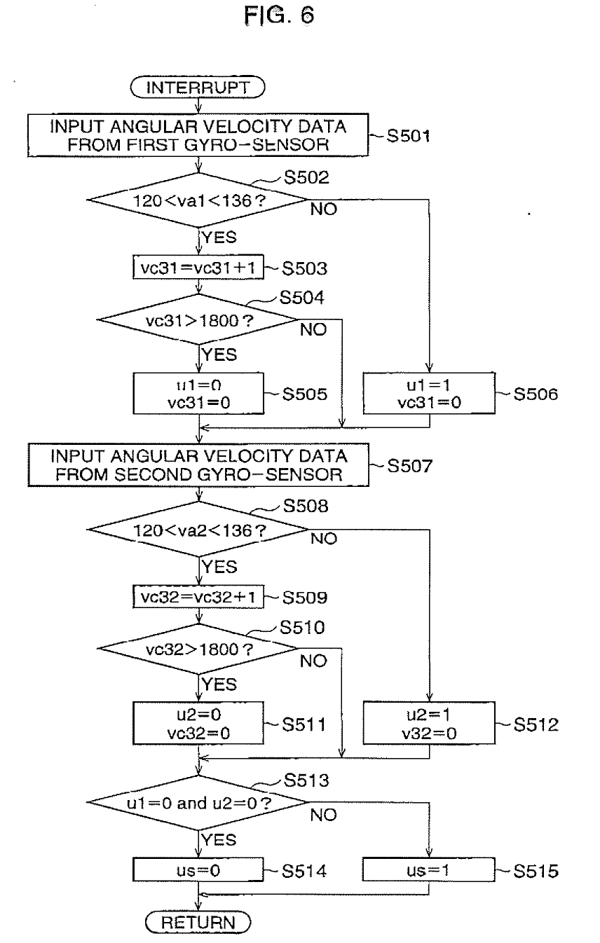

FIG. 6 is a flowchart of the usage state detection process performed by the scope controller 20. This process is carried out by interrupting a main routine (herein, not explained), which is performed by the scope controller 20, at 1/60 second intervals.

In Step S501, angular velocity data is input from the first gyro-sensor 22 to the scope-controller 20. The value of the angular velocity is herein represented by an integer in the range from 0 to 255. When the angular velocity is in the range from 121 to 135, it is determined by the first gyro-sensor 22 that the video-scope 10 is not moved. In Step S502, it is determined whether the angular velocity is greater than 120 and smaller than 136.

When it is determined that the angular velocity is greater than 120 and smaller than 136, the process goes to Step S503. In Step S503, a timer variable “vc31” is incremented by 1. The timer variable “vc31” is a variable for measuring the time, in 1/60 second intervals, that the first gyro-sensor 22 does not detect motion of the video-scope 10. In Step S504, it is determined whether the timer variable “vc31” exceeds 1800; namely, whether the time that the video-scope 10 is stationary continues for more than 30 seconds.

When it is determined at Step S504 that the timer variable “vc31” does not exceed 1800, the process skips to Step S507. On the other hand, when it is determined in Step S504 that the timer variable “vc31” exceeds 1800, the process goes to Step s505. In Step S505, a motion variable “ul” set to 0, and the timer variable “vc31” is set to 0. The motion variable “u1” is a variable that represents the motion of the video-scope 10 detected by the first gyro-sensor 22. The motion variable “u1” is set to 0 when the video-scope 10 is stationary, whereas the motion variable “ul” is set to 1 when the video-scope 10 is in motion.

On the other hand, when it is determined at Step S502 that the angular velocity is equal to or smaller than 120, or is equal to or greater than 136, the process goes to Step S506. In Step S506, the motion variable “u1” is set to 1, and the timer variable “vc31” is set to 0.

In Step S507, angular velocity data is input from the second gyro-sensor 24. In Steps S508 to S512, it is determined by the second gyro-sensor 24 whether the video-scope 10 is in motion. If a situation in which the video-scope 10 is not in motion continues for 30 seconds, a motion variable “u2”, which represents the motion of the video-scope 10 detected by the second gyro-sensor 24, is set to 0. On the other hand, the motion variable “u2” is set to 1 if the video-scope 10 is in motion.

In Step S513, it is determined whether both of the motion variables “u1” and “u2” are 0; namely, whether the video-scope 10 is not in motion or is fixed (for example, whether the video-scope 10 is held by a scope-holder). When it is determined that both of the motion variables “ul” and “u2” are 0, the process goes to Step S514. In Step S514, the usage state variable “us”, as described above, is set to 0. On the other hand, when it is determined that the motion variable “u1” or the motion variable “u2” is not 0, the process goes to Step S515, where the usage state variable “us” is set to 1. The usage state variable “us” is periodically transmitted to the video-processor 30 where the value of the “usage state variable” defined in the video-processor 30 is set to the value of usage state variable “us” in the video-scope 10 (see Step S303 in FIG. 4).

In this way, in the first embodiment, as shown in FIG. 5, the auto-brightness adjustment process is carried out by controlling the stop 31 at 1/60 second intervals, and the data of the opening-degree of the stop 31, which varies with the brightness, is stored in the RAM 49 at one-second intervals to generate the distribution data of detected opening-degrees (S406). Then, the first ratio “al”, indicating the ratio of the number of incidences of relatively large opening-degree to the total detected number of incidences of opening-degrees, and the second ratio “as”, indicating the ratio of the number of incidences of relatively small opening-degree to the total detected number of incidences of opening-degree, are calculated, respectively, and are compared to the reference ratio of the large opening-degree “CAL” and to the reference ratio of the small opening-degree “CAS”, respectively (S411, S412 and S414). When the first ratio “al” is higher than the reference ratio “CAL”, or the second ratio “as” is higher than the reference ratio “CAS”, it is determined that the illuminating-light irradiated on the observed portion is aberrant, and character information for warning that the illuminating-light is aberrant is displayed on the monitor 70 (S413 and S415).

The values of first ratio “al” and the second ratio “as” may be optionally set. For example, a ratio of the number of incidences of relative large opening-degree, which is higher than 75 percent of the full opening-degree, to the total detected number of incidences of opening-degrees, may be set as the first ratio “al”. On the other hand, a ratio of the number of incidences of relative small opening-degree, which is lower than 25 percent of the full opening-degree, to the total detected number of incidences of opening-degrees, may be set as the second ratio “as”. Also, the values of the reference ratios “CAL” and “CAS”, respectively, may be optionally set in accordance with the connected video-scope. For example, the values of the reference ratios “CAL” and “CAS” may be set to ¾ and ¼. The information that the illuminating light is aberrant may be reported aurally, such as, by a buzzer sound.

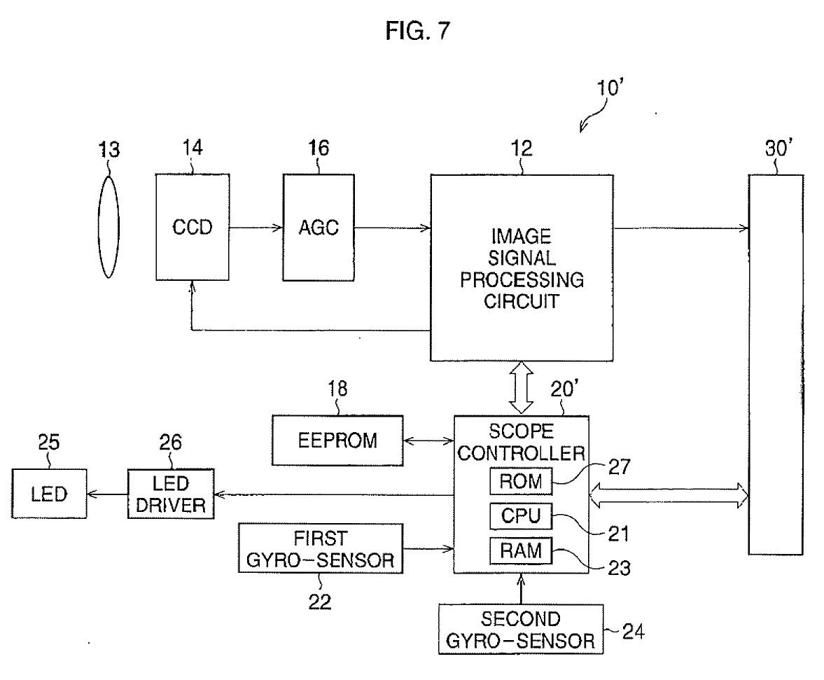

With reference to FIGS. 7 to 9, a second embodiment is explained. The second embodiment is different from the first embodiment in that the auto brightness adjustment process is performed by adjusting the amount of light emitted from a light source directly. Other constructions are substantially the same as those of the first embodiment.

FIG. 7 is a block diagram of an electronic endoscope according to the second embodiment.

A video-scope 10′ has an LED 25 and an LED driver 26. A scope-controller 20′ controls the video-scope 10′. In a ROM 27, a program for controlling the video-scope 10′ is stored. A stop and a lamp are not provided in a video-processor 30′, unlike in the first embodiment.

The LED 25, provided in the tip portion of the video-scope 10′, emits light in accordance with an electric current from the LED driver 26. The scope-controller 20′ controls the amount or electric current for the LED 25 on the basis of luminance signals fed from the image signal processing circuit 12, so that the brightness of the object image is maintained at a proper brightness.

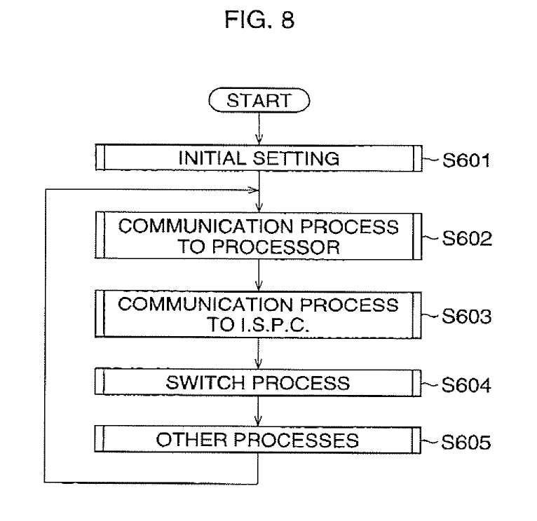

FIG. 8 is a flowchart of the main routine performed by the scope-controller 20′. In the second embodiment, the scope-controller 20′ (not the video-processor) detects any unusual or aberrant state of the illuminating light, unlike in the first embodiment.

In Step S601, an initial setting process is performed. In step S602, a process of communication with the video-processor 30′ is performed. In Step S603, a process of communication with the image signal processing circuit 12 is performed. In Step S604, a switch process associated with switches provided on the video-scope 10′ is performed. In Step S605, other processes are performed. Steps S602 to S605 is repeatedly performed until the video-scope 10′ is detached from the video-processor 30′, or the main electric power is turned OFF.

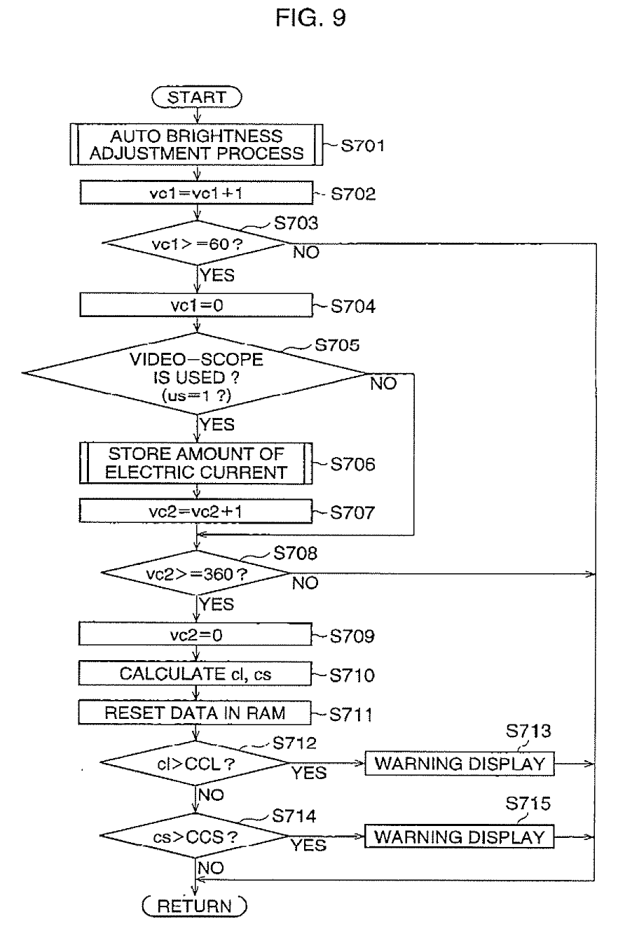

FIG. 9 is a flowchart of an auto-brightness; adjustment process and a self-diagnosis process according to the second embodiment.

In step S701, the auto brightness adjustment process is performed. Namely, based on the difference (or ratio) between the detected luminance level of the object image and the reference luminance level, the amount of electric current supplied from the LED driver 26 to the LED 25 is adjusted such that the brightness of the object image is maintained at a proper brightness. The process from Steps S702 to S705 is the same as that from steps S402 to S405 shown in FIG. 5.

In Step S706, data of the amount of electric current, which is presently set by the scope controller 20′, is stored in the RAM 23. Herein, the value of the electric current is an integer in the range from 1 to 240. Similarly to in the first embodiment, the data of the amount of electric current is stored as frequency distribution data, which is divided into seven grades; a section from 1 to 39, a section from 40 to 79, a section from 80 to 119, a section from 120 to 159, a section from 160 to 199, a section from 200 to 219, and a section from 220 to 240.

The process from Steps S707 to S709 is the same as that from Steps S407 to S409 shown in FIG. 5. In Step S710, based on the frequency distribution data of the amount of electric current, which has been stored in the RAM 23, a third ratio “c1” and a fourth ratio “cs” are calculated. Note that the frequency distribution data is obtained during the six minutes that the video-scope 10 is substantially used. The third ratio “cl” represents a ratio of the number of incidences of a relatively high amount of electric current to the total detected number of incidences of electric current; namely, the ratio of detected number of times of relatively high amount of electric current to the total detected number of times of electric current during the six minutes (=360). Herein, the third ratio “cl” indicates the proportion of the seventh grade, the section from 220 to 240. On the other hand, the fourth ratio “cs” represents a ratio of the number of incidences of relatively low amount of electric current to the total detected number of incidences of electric current. Herein, the fourth ratio “cs” indicates the proportion of the first grade, the section from 1 to 39. In Step S711, the frequency distribution data stored in the RAM 23 is reset to 0.

In Step S712, it is determined whether the third ratio “cl” exceeds a reference ratio “CCL” corresponding to a large electric current. Namely, it is determined whether the situation in which the amount of electric current is extremely large continues while the auto-brightness adjustment process is performed. The value of the reference ratio “CCL” is determined in accordance with the type of the video-scope 10. For example, in the case of the video-scope for the bronchi, the reference ratio “CCL” is set to 90 percent; in the case of the video-scope for the stomach, the reference ratio “CCL” is set to 95 percent; in the case of the video-scope for the colon, the reference ratio “CCL” is set to 85 percent.

When it is determined in Step S712 that the third ratio “cl” exceeds the reference ratio “CCL”, the process goes to Step S713. In step S713, the character signals are output from the CRTC 45 so as to display character information that reports an aberrant situation of the electronic endoscope to the operator. On the other hand, when it is determined that the third ratio “cl” does not exceed the reference ratio “CCL”, the process goes to Step S714.

In Step S714, it is determined whether the fourth ratio “cs” exceeds a reference ratio “CCS” corresponding to the small electric current. Herein in the case of the video-scope for the bronchi, the reference ratio “CCS” is set to 25 percent; in the case of the video-scope for the stomach, the reference ratio “CCS” is set to 25 percent; in the case of the video-scope for the colon, the reference ratio “CCS” is set to 35 percent. When it is determined that the fourth ratio “Cs” does not exceed the reference ratio “CCS”, the process terminated. On the other hand, when it is determined that the fourth ratio “cs” exceeds the reference ratio “CCS”, the process goes to Step S715, where the character signals are output from the CRTC 45 so as to display character information that reports an aberrant situation to the operator.

In this way, in the second embodiment, the auto brightness adjustment process is carried out by adjusting the amount or electric current fed from the LED driver 25 to the LED 26, and the self-diagnosis process is carried out on the basis of the amount of electric current. Then, as shown in FIG. 9, the third ratio “cl”, indicating a ratio of the number of incidences of a large value of electric current to the total detected number of incidences of electric current, and the fourth ratio “cs”, indicating a ratio of the number of incidences of a small value of electric current to the total detected number of incidences of electric current, are calculated, respectively, and are compared to the reference ratio “CCL” and to the reference ratio “CCS”, respectively (S710, S712 and S714), When the third ratio “cl” is greater than the reference ratio “CCL”, or the fourth ratio “cs” is greater than the reference ratio “ccs”, it is determined that the illuminating-light irradiated on the observed portion is in aberrant state, and character information for warning that the illuminating-light is aberrant is displayed on the monitor 70 (S713 and S715).

The third ratio “cl” and the fourth ratio “cs” may be optionally set. For example, a ratio of the number of incidences of relatively high value of electric current, which is higher than 75 percent of the maximum value of electric current to the total detected number of incidences of electric current, may be set as the third ratio “cl”. On the other hand, a ratio of the number of incidences of a relatively low value of electric current, which is smaller than 25 percent of the maximum value of electric current, to the total detected number of incidences of electric current may be set as the second ratio “cs”. Also, the values of the reference ratios “CCL” and “CCS”, respectively, may be optionally set in accordance with the connected video-scope. For example, the values of the reference ratios “CCL” and “CCS” may be set to ¾ and ¼, respectively.

The LED 25 may be installed in the video-processor. In this case, the video-processor may adjust the amount of electric current. The auto-brightness adjustment process and the self-diagnosis process may be carried out in a light source unit used for a fiber-scope. Another light source may be used instead of an LED, and may adjust the amount of electric current associated with the amount of illuminating-light.

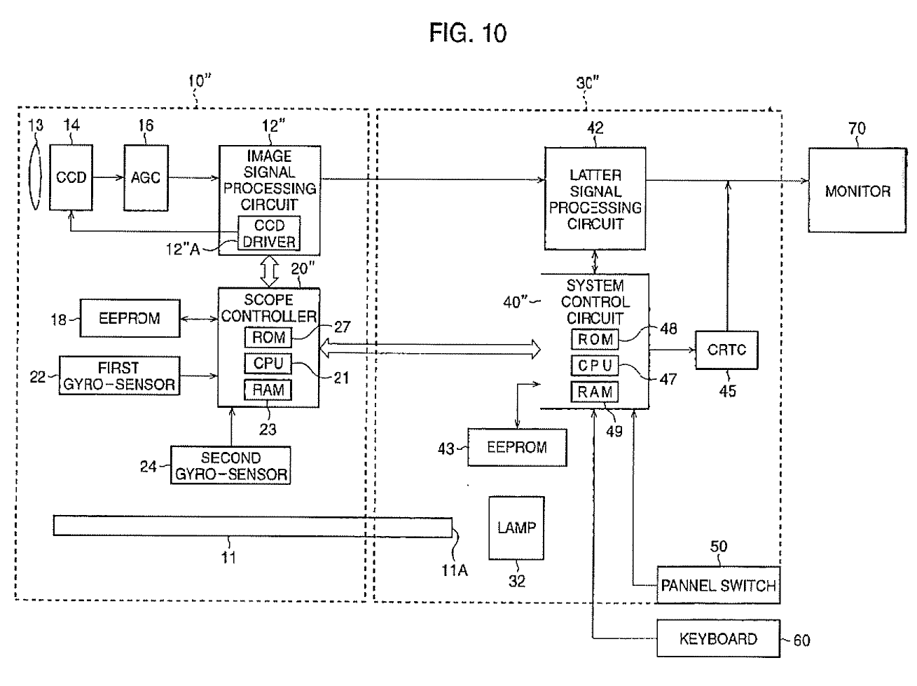

With reference to FIGS. 10 to 13, a third embodiment is explained. The third embodiment is different from the first and embodiments in that the auto brightness adjustment process is performed by an electronic shutter function. Other constructions are substantially the same as those of the first embodiment and the second embodiment.

FIG. 10 is a block diagram of an electronic endoscope according to the third embodiment.

A scope-controller 20″ in a video-scope 10″ carries out the auto brightness adjustment process on the basis of luminance signals detected by an image signal processing circuit 12″. The scope-controller 20″ outputs control signals to the image signal processing circuit 12″, to set a charge-accumulation period, or an electronic shutter speed. The CCD 14 is driven by driving signals fed from the CCD driver 12″A such that image-pixel signals are read from the CCD 14 in accordance with the determined charge-accumulation period. Thus, the proper brightness off the object image is maintained.

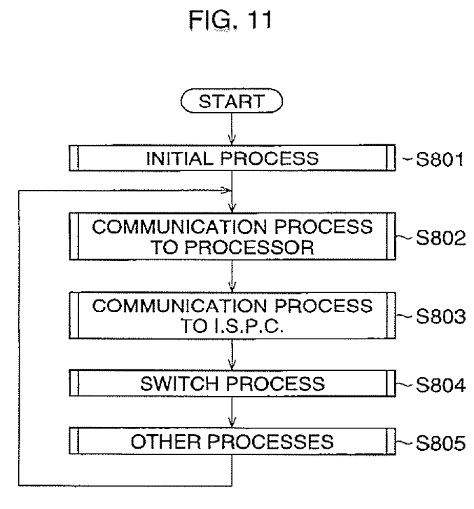

FIG. 11 is a view showing a main routine performed by the scope-controller 20″. The process from Steps S801 to S805 is the same as that from Steps S601 to S605 shown in FIG. 8.

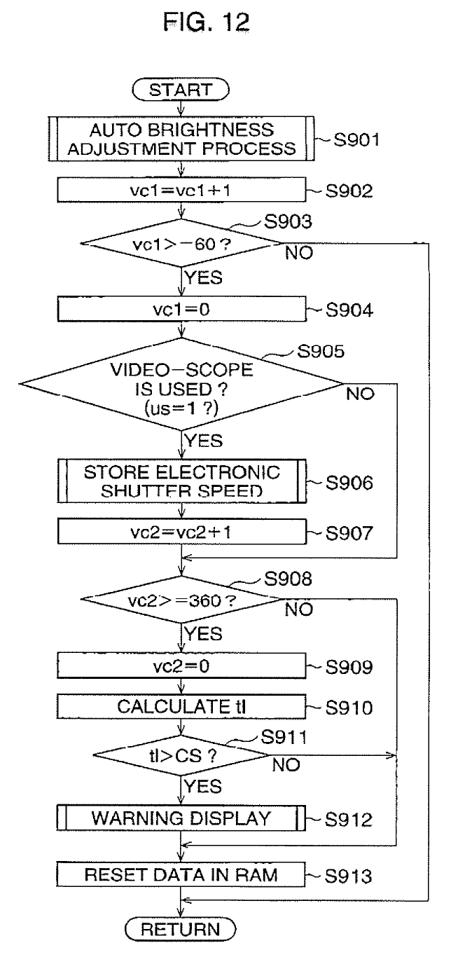

FIG. 12 is a view showing a flowchart of an auto-brightness adjustment process and a self-diagnosis process according to the third embodiment. This process is carried out by interrupting the main routine shown in FIG. 11 at 1/60 second intervals,

In Step S901, based on the difference between the detected luminance level of the object image and the reference luminance level, the electronic shutter speed or the charge-accumulation period is adjusted such that the proper brightness of the object imago is maintained. The process from Steps S902 to s905 is the same as that from Steps S702 to S705 shown in FIG. 9.

In Step S906, data on the electronic shutter speed, which is presently set by the scope controller 20″, is stored in the RAM 23. Herein, the value of the electronic shutter speed is a value in the range from 1/60 to 1/10000. The data of the electronic shutter speed is stored as frequency distribution data, which is divided into eight grades; a section from 1/60 to less than 1/80, a section from 1/80 to less than 1/120, a section from 1/120 to less than 1/250, a section from 1/250 to less than 1/500, a section from 1/500 to less than 1000, a section from 1/1000 to less than 1/2000, a section from 1/2000 to less than 1/4000, and a section from 1/4000 to 1/40000. The process from Steps S907 to S909 is the same as that from Steps S707 to S709 in FIG. 9.

In Step S910, based on the frequency distribution data for the electronic shutter speed, which has been stored in the RAM 23, a fifth ratio “tl” is calculated. Note that, the frequency distribution data is obtained over the six minutes that the video-scope 10 is substantially used. The fifth ratio “tl” represents a ratio of the number of incidences of relatively low speed, indicated by high value of electronic shutter speed, to the total detected number of incidences of electronic shutter speeds. Herein, the fifth ratio “tl” indicates the proportion of the first grade, the section from 1/60 to less than 1/80.

In Step S911, it is determined whether the fifth ratio “tl” exceeds a reference ratio “CS” corresponding to the low speed. Namely, it is determined whether the situation where the electronic shutter speed is extremely high continues while the auto-brightness adjustment process is performed. For example, in the case of the video-scope for the bronchi, the reference ratio “CS” is set to 90 percent.

When it is determined that the third ratio “tl” does not exceed the reference ratio “CS”, the process skips to Step S913. On the other hand, when it is determined at Step S910 that the fifth ratio “tl” exceeds the reference ratio “CS”, the process goes to Step S912. In Step S912, the character signals are output from the CRTC 45 so as to display character information that reports an aberrant situation of the electronic endoscope to the operator. In Step S913′, the frequency distribution data stored in the RAM 23 is reset to

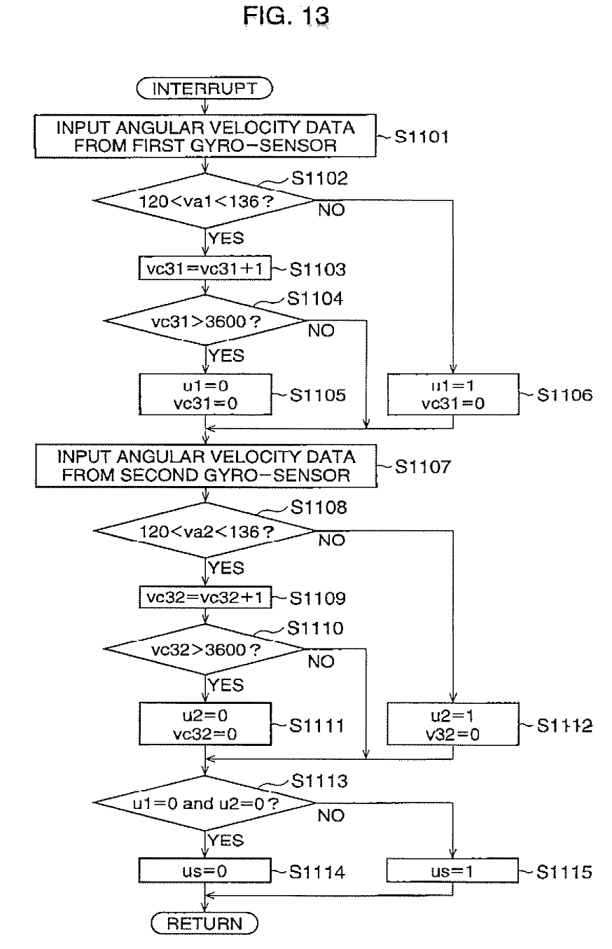

FIG. 13 is a flowchart of a usage state detection process according to the third embodiment. The process from Steps S1101 to S1115 is the same as that from Steps S501 to S515 shown in FIG. 6.

In this way, in the third embodiment, the auto-brightness adjustment process is carried out, and the self-diagnosis process is carried out on the basis of the electronic shutter speed. Then, the fifth ratio “tl”, indicating a ratio of the number of incidences of low electronic shutter speed to the total detected number of incidences of electronic shutter speed is calculated, and is compared to the reference ratio “CS” (S910 and S911). When the fifth ratio “tl” is higher than the reference ratio “CS”, it is determined that the illuminating-light irradiated on the observed portion is in aberrant state, and character information for warning that the illuminating-light is aberrant is displayed on the monitor 70 (S912).

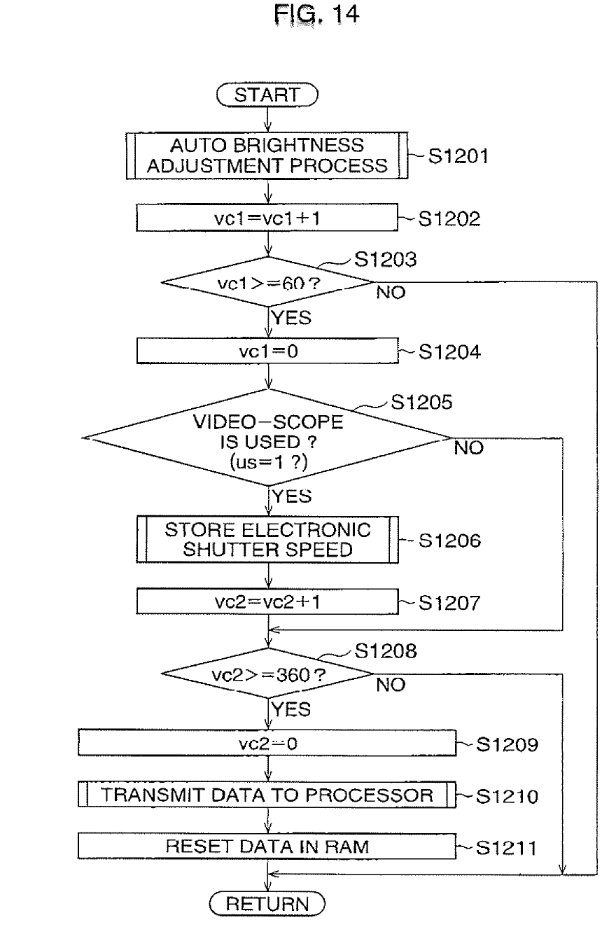

With reference to FIGS. 14 to 17, a fourth embodiment is explained. The fourth embodiment is different from the first, second, and third embodiments in that the video-processor detects an amount of illuminating light on the basis of electronic shutter speed data. Other constructions are substantially the same as those of the first, second, and third embodiments.

FIG. 14 is a flowchart or an auto brightness adjustment process and a self-diagnosis process according to the fourth embodiment. This process is carried out by the scope-controller 20″ and interrupts the main routine, carried out by the scope-controller 20″, at 1/60 second intervals.

The process from Steps S1201 to S1209 is the same as that from Steps S901 to S909 shown in FIG. 12. Namely, the data of electronic shutter speed is stored in the RAM 23 at one-second intervals. In Step S1210, data of a series of detected electronic shutter speed is transmitted to the system control circuit 40″ at six minute intervals. In Step S1211, the data stored in the RAM 23 is reset to 0.

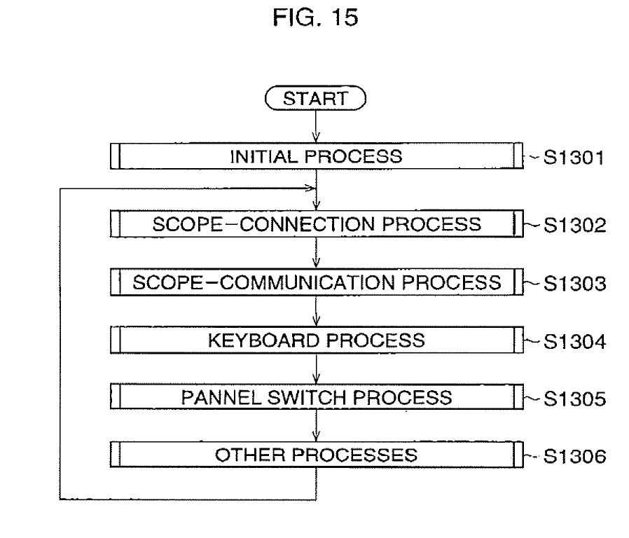

FIG. 15 is a view showing a main routine performed by the system control circuit 40″. The process from Steps S1301 to S1306 is the same as that from Steps S101 to S106 shown in FIG. 2.

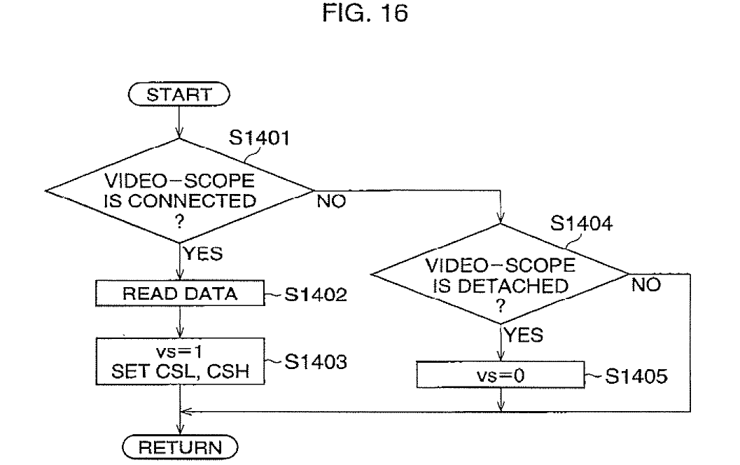

FIG. 16 is a view showing a subroutine of Step S1302 shown in FIG. 15. The process from Steps S1401 to S1405 is the same as that from Steps S201 to S205 shown in FIG. 3. Note that, in Step S1403, reference ratios “CSL” and “CSH”, described later, are set.

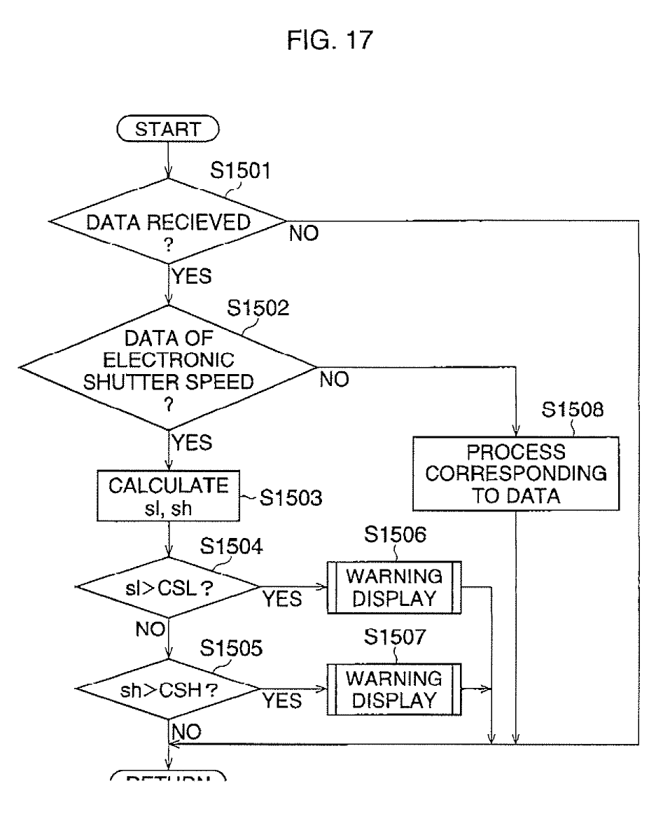

FIG. 17 is a view showing a subroutine of Step S1303 shown in FIG. 15.

In Step S1501, it is determined whether data has been transmitted from the video-scope 10″. When it is determined that the data has not been transmitted from the video-scope 10″, the process is terminated. On the other hand, when it is determined that the data has been transmitted from the video-scope 10″, the process goes to Step S1502. In Step S1502, it is determined whether the transmitted data is data on the electronic shutter speed.

When it is determined that the transmitted data is not data of the electronic shutter speed, the process goes to Step S1508. In Step S1508, a process corresponding to the data is performed. On the other hand, when it is determined that the transmitted data is data of the electronic shutter speed, the process goes to Step S1503. In Step S1503, based on the frequency distribution data of the electronic shutter speeds, which has been stored in the RAM 23, a sixth ratio “sl” and a seventh ratio “sh” are calculated. The sixth ratio “s1” represents a ratio of the number of incidences of relatively low shutter speed (high value of the electronic shutter speed) to the total detected number of incidences of electronic shutter speeds. On the other hand, the seventh ratio “sh” represents a ratio of the number of incidences of relatively high speed (low value of electronic shutter speed) to the total detected number of incidences of electronic shutter speeds. Herein, the sixth ratio “sl” indicates the proportion of the first grade, the section from 1/60 to less than 1/80, whereas the seventh ratio “sh” indicates the proportion of the seventh grade, the section from 1/2000 to 1/40000.

In Step S1504, it is determined whether the sixth ratio “sl” exceeds the reference ratio “CSL” determined at Step S1403 in FIG. 16. Namely, it is determined whether the situation in which the electronic shutter speed is extremely low continues while the auto-brightness adjustment process is performed. For example, in the case of the video-scope for the colon, the reference ratio “CSL” is set to 85 percent. When it is determined that the sixth ratio “sl” exceeds the reference ratio “CSL”, the process goes to Step S1506. In Step S1506, the character signals are output from the CRTC 45 so as to display character information that reports an aberrant situation of the electronic endoscope to the operator.

On the other hand, when it is determined that the sixth ratio “sl” does not exceed the reference ratio “CSL”, the process goes to step S1505, In Step S1505, it is determined whether the seventh ratio “sh” exceeds the reference ratio “CSH” determined at Step S1403 in FIG. 16. Namely, it is determined whether the situation in which the electronic shutter speed is extremely high continues while the auto-brightness adjustment process is performed. For example, in the case of the video-scope for the colon, the reference ratio “CSH” is set to 5 percent. When it is determined that the seventh ratio “sh” does not exceed the reference ratio “CSH” the process is terminated. On the other hand, when it is determined that the seventh ratio “sh” exceeds the reference ratio “CSH” the process goes to Step S1507, where the character signals are output from the CRTC 45 so as to display character information that reports an aberrant situation.

In this way, in the fourth embodiment, the auto brightness adjustment process is carried out by adjusting the electronic shutter speed, and the self-diagnosis process is carried out in the video-processor. Then, the sixth ratio “sl” corresponding to the low shutter speed and the seventh ratio “sh” corresponding to the high shutter speed are calculated, respectively, and are compared to the reference ratio “CSL” and the reference ratio “CSH”, respectively (S1503, S1504 and Sl505). When the sixth ratio “sl” is higher than the reference ratio “CSL”, or the seventh ratio “sh” is higher than the reference ratio “CSH”, it is determined that the illuminating-light irradiated on the observed portion is in an aberrant state, and character information for warning that the illuminating-light is aberrant is displayed on the monitor 70 (S1506 and S1507).

The sixth ratio “sl” and the seventh ratio “sh” may be set, optionally. For example, a ratio of the number of incidences of relatively low shutter speed, which is lower than 10 percent to the maximum shutter speed, to the total detected number of incidences of electronic shutter speeds, may be set as the sixth ratio “sl”. On the other hand, a ratio of the number of incidences of relatively high shutter speed, which is higher than 80 percent to the maximum shutter speed, to the total detected number of incidences of electronic shutter speeds, may be set as the seventh ratio “sh”. Note that, the maximum shutter speed is represented by logarithm function when setting the sixth and seventh ratios. Further, the values of the reference ratios “CSL” and “CSH”, respectively, may be set in accordance with the connected video-scope. For example, the values of the reference ratios “CSL” and “CSH” may be set to ¾ and ¼, respectively.

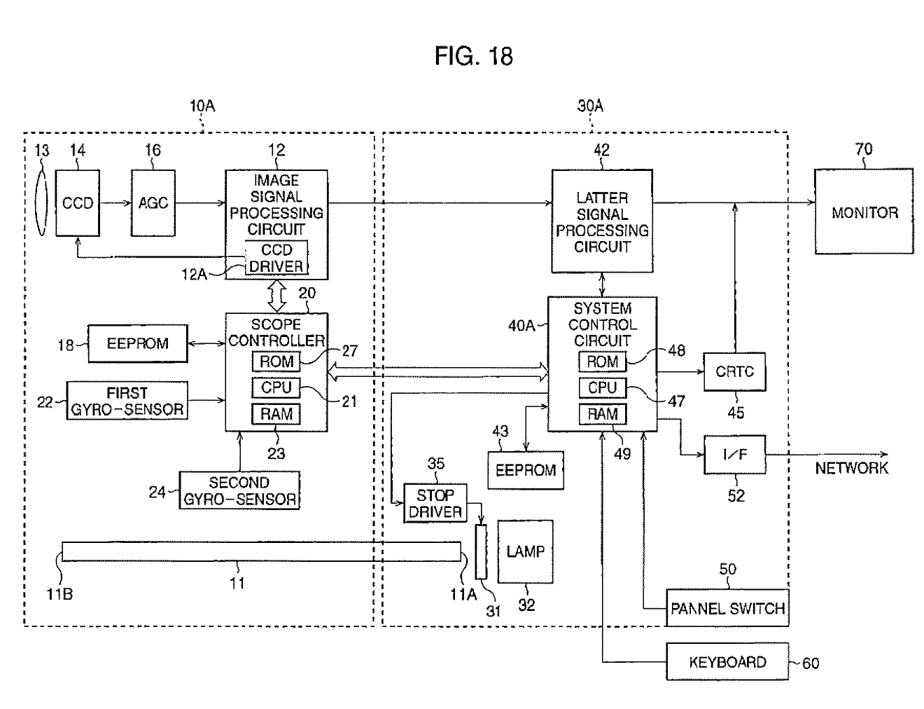

With reference to FIGS. 18 to 20, a fifth embodiment is explained. The fifth embodiment is different from the first embodiment in that an aberrant state of the electronic endoscope is transmitted to an outside apparatus, such as, a computer via network communication; and a maximum amount of illuminating light is detected. Other constructions are substantially the same as those of the first embodiment.

FIG. 18 is a block diagram of an electronic endoscope according to the fifth embodiment.

A system control circuit 40A provided in a video-processor 30A is connected to outside equipment, such as a computer, by a network cable. The data is transmitted between the video-processor 30A and the outside equipment via an interface circuit (I/F) 52. Herein, the video-processor 30A sends the data via e-mail messages.

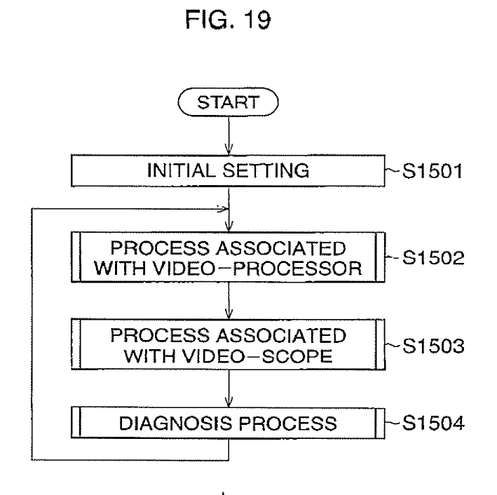

FIG. 19 is a view showing a main routine performed by the system control circuit 40A.

In Step S1501, the initial setting process is performed. Further, the system control circuit 40A detects whether any system error occurs when starting the electronic endoscope. In Step S1502, a process associated with the video-processor 30 is performed. In Step S1503, a process associated with the video-scope 10 is performed. In Step S1504, a process of self-diagnosis is performed.

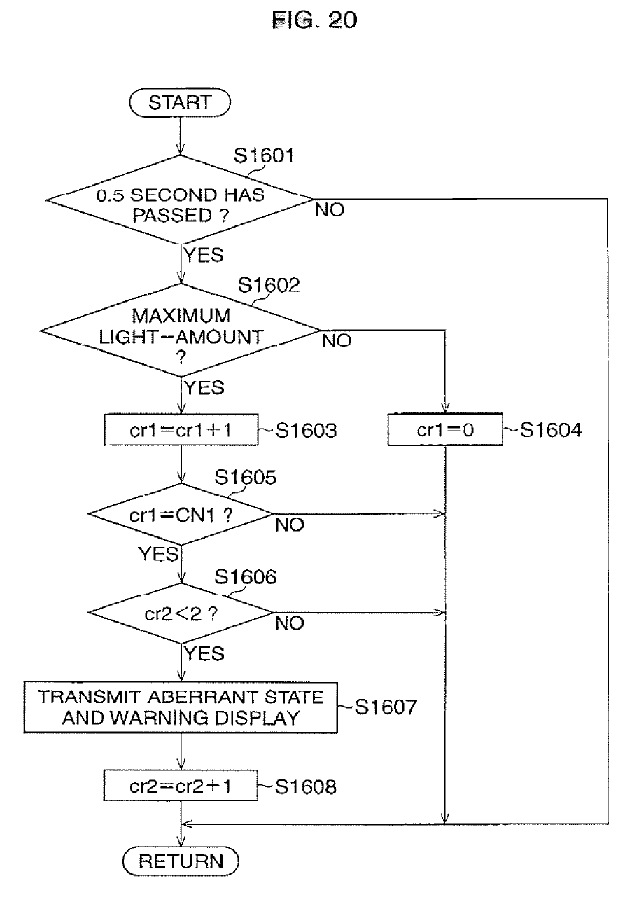

FIG. 20 is a view showing a subroutine of Step S1504 shown in FIG. 19.

In Step S1601, it is determined whether 0.5 seconds have elapsed since the previous diagnosis process. Herein, the amount of illuminating light is detected at 0.5 second intervals by detecting the opening-degree of the stop 31. When it is determined that 0.5 seconds have not elapsed since the previous diagnosis process, the process is terminated. On the other hand, when it is determined that 0.5 second has passed from the previous diagnosis process; the process goes to Step S1602.

In Step S1602, it is determined whether the stop 31 fully opens; namely, whether the amount of illuminating light is a maximum amount. When it is determined that the stop 31 fully opens, the process goes to Step S1603. In Step S1603, a timer variable “cr1” is incremented by 1. The timer variable “crl” is a variable for measuring an interval that the maximum illuminating light is maintained. On the other hand, when it is determined that the stop 31 does not fully open, the process goes to Step S1604. In Step S1604, the timer variable “cr1”0 is set to 0. Note that the timer variable is initially set to 0 at Step S1501 shown in FIG. 19.

In Step S1605, it is determined whether the timer variable “crl” equals a constant “CN1”. Namely, the status of maximum illuminating light continues for a given interval. Herein, the constant “CN1” is set to 120. Thus, in Step S1605, it is determined whether the status of maximum illuminating light continues for 60 seconds. In this embodiment, if the maximum amount of illuminating light continues for 60 seconds, it is determined that the auto-brightness adjustment process is in an aberrant state. When it is determined that the timer variable “cr1” does not equal the constant “CN1”, the process is terminated. On the other hand, when it is determined that the timer variable “cr1” equals the constant “CN1”, the process goes to Step S1606.

In Step S1606, it is determined whether a reporting restriction variable “cr2” is lower than 2. The reporting restriction variable “cr2” is a variable for restricting the number of times that the aberrant state is reported outside. Herein, the information is transmitted outside twice. When it is determined that the reporting restriction variable “cr2” is not smaller than 2, the process is terminated. On the other hand, when it is determined that the reporting restriction variable “cr2” is smaller than 2, the process goes to Step S1607. Note that, the reporting restriction variable “cr2” is set to 0 at the initial setting process.

In Step S1607, data that reports an aberrant state of the electronic endoscope is transmitted from the video-processor 30 to an outside computer system provided in a repair center. At this time, data associated with the electronic endoscope, such as the endoscope name and a registration number, is simultaneously transmitted. Also, to report the aberrant state to the operator, character signals are output to the CRTC 45 so as to display character information that warns against use of the electronic endoscope. In Step S208, the reporting restriction variable “cr2” is incremented by 1.

In this way, in the fifth embodiment, the auto-brightness adjustment process is carried out by adjusting the opening-degree of the stop, and the self-diagnosis process is carried out on the basis of the opening-degree. If a situation in which the opening-degree is at the maximum continues for 60 seconds, it is determined that the illuminating-light is in an aberrant state, and data that reports the aberrant state is transmitted to the outside equipment via the network (S1607).

The interval over which the maximum opening-degree continues may be optionally set to another interval, instead of to 60 seconds. Also, when a situation in which the amount of illuminating light in the range of 70 percent to 100 percent of the maximum amount of illuminating-light continues for a given interval, it may be determined that the illuminating-light is in an aberrant state.

Finally, it will be understood by those skilled in the arts that the foregoing description is of preferred embodiments of the device, and that various changes and modifications may be made to the present invention without departing from the spirit and scope thereof.

The present disclosure relates to subject matter contained in Japanese Patent Applications No. 2005-34346l (filed on Nov. 29, 2005), No. 2005-343531 (filed on Nov. 29, 2005), No. 2005-343660 (filed on Nov. 29, 2005), and No. 2005-343704 (filed on Nov. 29, 2005), which are expressly incorporated herein, by reference, in their entireties.

1. An electronic endoscope comprising:
a video-scope with an image sensor;
a light source that radiates illuminating-light on an object;
a brightness adjuster that maintains a brightness of an object image at a proper brightness on the basis of an amount of illuminating-light;
a brightness state detector that detects whether the amount of illuminating-light is aberrant while the brightness is adjusted by said brightness adjuster; and
a reporting processor that reports an aberrant state of the illuminating light.

2. The electronic endoscope of claim 1, wherein said brightness state detector measures the amount of illuminating-light periodically.

3. The electronic endoscope of claim 1, wherein said brightness state detector determines the aberrant state if a state in which the amount of illuminating-light exceeds an amount close to a maximum amount continues for a given interval.

4. The electronic endoscope of claim 1, wherein said brightness state detector measures the amount of illuminating-light at regular intervals to obtain a distribution of amounts of illuminating-light, said brightness state detector determining the aberrant state if a proportion of a relatively large amount of illuminating-light in the distribution exceeds a given proportion.

5. The electronic endoscope of claim 1, wherein said brightness state detector detects the aberrant state in a period in which the electronic endoscope is used for endoscope work.

6. The electronic endoscope of claim 1, further comprising a stop that opens and closes to adjust the amount of illuminating-light, said brightness adjuster adjusting an opening-degree of said stop on the basis of image-pixel signals read from said image sensor,
wherein said brightness state detector measures the opening-degree of said stop, and detects the aberrant state on the basis of the measured opening-degree.

7. The electronic endoscope of claim 6, wherein said brightness state detector determines the aberrant state if a state of a relatively large opening-degree continues for a given interval.

8. The electronic endoscope of claim 6, wherein said brightness state detector determines the aberrant state if a state of relatively small opening-degree continues for a given interval.

9. The electronic endoscope of claim 6, wherein said brightness state detector stores the measured opening-degree in a memory at regular intervals to obtain a distribution of opening-degrees, and determines the aberrant state on the basis of the distribution or opening-degree.

10. The electronic endoscope of claim 6, wherein said brightness state detector measures the opening-degree periodically to obtain a distribution of opening-degrees, and determines the aberrant state if a ratio of the number of incidences of a relatively large opening-degree to the total detected number of incidences of opening-degrees exceeds a given ratio.

11. The electronic endoscope of claim 6, wherein said brightness state detector measures the opening-degree periodically to obtain a distribution of opening-degrees, and determines the aberrant state if a ratio of the number of incidences of a relatively small opening-degree to the total detected number of incidences of opening-degrees exceeds a given ratio.

12. The electronic endoscope of claim 6, further comprising a usage state detector that detects whether said video-scope is used for performing endoscope work, said brightness state detector measuring the opening-degree only when said video-scope is used.

13. The electronic endoscope of claim 1, further comprising a light source driver that drives said light source by supplying electric current to said light source said brightness adjuster adjusting an amount of electric current on the basis of image-pixel signals read from said image sensor,
wherein said brightness state detector measures the amount of electric current, and detects the aberrant state on the basis of the measured amount of electric current.

14. The electronic endoscope of claim l3, wherein said brightness state detector determines the aberrant state if a state of relatively large amount of electric current continues for a given interval.

15. The electronic endoscope of claim 13, wherein said brightness state detector determines the aberrant state if a status of relatively small amount of electric current continues for a given interval.

16. The electronic endoscope of claim 13, wherein said brightness state detector stores the measured opening-degree in a memory at regular intervals to obtain a distribution of amounts of electric current, and determines the aberrant state on the basis of the distribution of amounts of electric current.

17. The electronic endoscope of claim 13, wherein said brightness state detector measures the amount of electric current periodically to obtain a distributions of amounts of electric current, and determines the aberrant state if a ratio of the number of incidences of a relatively large amount of electric current to the total detected number of incidences of amounts of electric current exceeds a given ratio.

18. The electronic endoscope of claim 13, wherein said brightness state detector measures the amount of electric current periodically to obtain a distribution of amounts of electric current, and determines the aberrant state if a ratio of the number of incidences of a relatively small amount of electric current to the total detected number or incidences of amounts of electric current exceeds a given ratio.

19. The electronic endoscope of claim 13, further comprising a usage state detector that detects whether said video-scope is used for performing endoscope work, said brightness state detector measuring the amount of electric current only when said video-scope is used.

20. The electronic endoscope of claim 1, wherein said brightness adjuster maintains the proper brightness by adjusting an electronic shutter speed of said image sensor on the basis of image-pixel signals read from said image sensor,
wherein said brightness state detector measures the electronic shutter speed, and detects the aberrant state on the basis of the measured electronic shutter speed.

21. The electronic endoscope of claim 20, wherein said brightness state detector determines the aberrant state if a state of relatively low shutter speed continues for a given interval.

22. The electronic endoscope of claim 20, wherein said brightness state detector determines the aberrant state if a status of relatively high shutter speed continues for a given interval.

23. The electronic endoscope of claim 20, wherein said brightness state detector stores the measured electronic shutter speed in a memory at regular intervals to obtain a distribution of electronic shutter speeds, and determines the aberrant state on the basis of the distribution of electronic shutter speeds.

24. The electronic endoscope of claim 20, wherein said brightness state detector measures the electronic shutter speed periodically to obtain a distribution of electronic shutter speeds, and determines the aberrant state if a ratio of the number of incidences of a relatively low shutter speed to the total detected number of incidences of electronic shutter speeds exceeds a given ratio.

25. The electronic endoscope of claim 20, wherein said brightness state detector measures the electronic shutter speed periodically to obtain a distribution of electronic shutter speeds, and determines the aberrant state if a ratio of the number of incidences of a relatively high shutter speed to the total detected number of incidences of electronic shutter speeds exceeds a given ratio.

26. The electronic endoscope of claim 20, further comprising a usage state detector that detects whether said video-scope is used for performing endoscope work, said brightness state detector measuring the electronic shutter speed only when said video-scope is used.

27. The electronic endoscope of claim 1, wherein said reporting processor transmits data associated with the aberrant state to outside equipment.

28. The electronic endoscope of claim 27, wherein said brightness state detector detects whether a state in which the amount of illuminating-light is substantially maximum continues for a given interval.

29. The electronic endoscope of claim 27, wherein said reporting processor transmits data associated with the aberrant state by one or twice.

30. An apparatus for diagnosing an electronic endoscope, comprising:
a brightness state detector that detects whether an amount of illuminating-light is aberrant, while an brightness adjuster maintains a brightness of an object image at a proper brightness on the basis of an amount of illuminating-light; and
a reporting processor that reports an aberrant state of the illuminating light.

31. The apparatus of claim 30, wherein said brightness adjuster maintains the proper brightness by adjusting an opening-degree of a stop on the basis of image-pixel signals read from an image sensor provided in a video-scope,
wherein said brightness state detector measures the opening-degree of said stop, and detects the aberrant state on the basis of the measured opening-degree.

32. The apparatus of claim 30, wherein said brightness adjuster maintains the proper brightness by adjusting an amount of electric current supplied to a light source that radiates the illuminating-light on the basis of image-pixel signals read from an image sensor provided in a video-scope,
wherein said brightness state detector measures an amount of electric current, and detects the aberrant state on the basis of the measured amount of electric current.

33. The apparatus of claim 30, wherein said brightness adjuster maintains the proper brightness by adjusting an electronic shutter speed of said image sensor on the basis of image-pixel signals read from an image sensor provided in a video-scope,
wherein said brightness state detector measures the electronic shutter speed, and detects the aberrant state on the basis of the measured electronic shutter speed.

34. The apparatus of claim 30, wherein said reporting processor transmits data associated with the aberrant state to outside equipment.

35. A method for diagnosing an electronic endoscope, comprising:
detecting whether an amount of illuminating-light is aberrant while maintaining a brightness of an object image at a proper brightness on the basis of an amount of illuminating-light; and
reporting an aberrant state of the illuminating light.

36. The method of claim 35, wherein said maintaining comprises adjusting an opening-degree of a stop on the basis of image-pixel signals read from an image sensor provided in a video-scope,
wherein said detecting comprises measuring the opening-degree of said stop, and detecting the aberrant state on the basis of the measured opening-degree.

37. The method of claim 35, wherein said maintaining comprises adjusting an amount of electric current supplied to a light source that radiates the illuminating-light on the basis of image-pixel signals read from an image sensor provided in a video-scope,
wherein said detecting comprises measuring an amount of electric current, and detecting the aberrant state on the basis of the measured amount of electric current.

38. The method of claim 35, wherein said maintaining comprises adjusting an electronic shutter speed of said image sensor on the basis of image-pixel signals read from an image sensor provided in a video-scope,
wherein said detecting comprises measuring the electronic shutter speed, and detecting the aberrant state on the basis of the measured electronic shutter speed.

39. The method of claim 35, wherein said reporting comprises transmitting data associated with the aberrant state to outside equipment.
